(19) United States
(12) Patent Application Publication
(10) Pub. No.: US 2011/0008798 A1
(43) Pub. Date: Jan. 13, 2011

(54) Lipid insertion for antigen capture and presentation and use as a sensor platform

(76) Inventors: Harshini Mukundan, Los Alamos, NM (US); Anu Chaudhary, Los Alamos, NM (US); Basil I. Swanson, Los Alamos, NM (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(21) Appl. No.: 12/658,298

(22) Filed: Feb. 8, 2010

Related U.S. Application Data
(60) Provisional application No. 61/206,980, filed on Feb. 6, 2009.
(60) Provisional application No. 61/251,605, filed on Oct. 14, 2009.

(51) Int. Cl. G01N 33/569

(52) U.S. Cl. 435/7.1

(57) ABSTRACT

It has been found that moieties containing a lipophilic domain, e.g., lipophilic pathogen activated molecular patterns (PAMPs), insert into the lipid bilayer on a cell membrane to facilitate antigen recognition by the innate immune response receptors. This changes the basic understanding of antigen recognition by the innate immune system. A sensor platform for the ultra-sensitive and specific detection of moieties containing such a lipophilic domain, e.g., PAMPs, that are associated with a disease, has now been developed. To date, this approach has been validated with Lipoarabinomannan (LAM) from Mycobacterium tuberculosis and lipopolysacharide (LPS), associated with gram-negative bacteria. This approach may be extendable to all lipophilic targets associated with pathogens and thus, is the basis of a very simple and specific sensing platform. In addition, novel applications for this technology in the selection of recognition ligands by mass spectroscopy have been identified.

### CROSS REFERENCE TO RELATED APPLICATION

This patent application claims the benefit of the filing date of U.S. provisional patent application No. 61/206,980 filed Feb. 6, 2009 and provisional patent application No. 61/251,605 filed Oct. 14, 2009.

### STATEMENT OF FEDERAL RIGHTS

The United States government has rights in this invention pursuant to Contract No. DE-AC52-06NA25396 between the United States Department of Energy and Los Alamos National Security, LLC for the operation of Los Alamos National Laboratory.

### FIELD OF THE INVENTION

The present invention relates to methods for capture of target moieties containing a lipophilic domain and methods for identification and/or analysis following capture.

### BACKGROUND OF THE INVENTION

Host innate immune recognition receptors, i.e., Toll-Like Receptors (TLRs), exhibit extra-ordinary specificities for conserved molecular motifs, termed pathogen associated molecular patterns (PAMPs), found in many different classes of pathogens. There are ten different classes of human TLRs (see FIG. 1). PAMPs (e.g., LAM, bacterial LPS, peptidoglycan, viral nucleic acids, etc.) represent the molecular signature of a particular class of pathogen (Uematsu and Akira, 2008). Recognition of PAMPs by TLRs on a host cell triggers host defense mechanisms culminating in pathogen elimination. It has been perceived that PAMPs are recognized by the extracellular domains of TLRs, initiating signal transduction cascades that culminate in the production of immune mediators such as cytokines. However, the exact physiology of PAMPs recognition at the cellular level has not been delineated. Interestingly, many PAMPs have a lipid component to them.

Biomarkers are loosely defined as biomolecules that are differentially expressed during the course of disease. In the case of infectious disease, these are pathogen-associated biomolecules that are secreted in the host during infection. Many biomarkers are virulence factors required for pathogenicity of the infectious agent and several are expressed very early in disease onset. It is therefore logical to presume that detection of biomarkers can allow for the specific and early detection of disease. However, use of biomarkers in this regard is very limited, primarily because current detection strategies lack the sensitivity required of the detection of extremely low concentrations of these molecules in the infected host. Indeed, many of the current biomarker-based approaches (e.g., QuantiFERON®—TB Gold test for Mycobacterium tuberculosis from Cellestis Limited, Carnegie, Victoria, Australia), measure the host immune response to the biomarker, rather than the target molecule itself. Further, many conventional detection strategies are sensitive to non-specific interactions associated with complex patient samples such as serum and urine, further compromising assay sensitivity. Perhaps the most important limitation of all is the fact that no single biomarker can accurately predict disease. This is because expression of biomarkers varies during the course of the disease. Therefore, an ideal detection strategy should be capable of the simultaneous ultra-sensitive detection of a limited suite of such molecules in complex patient samples.

### SUMMARY OF THE INVENTION

The following applications of this approach are available:

1. The use of lipid insertion of PAMPs and related lipophilic biomarkers as a sensor platform for ultrasensitive and specific detection of pathogens. A single-reporter fluorescence assay for the detection of PAMPs based on their lipid insertion has been developed. Because of the orientation of antigen presentation, very high sensitivities of detection in complex fluids such as serum have been accomplished. This detection is achieved on a previously developed waveguide-based biosensor (see, e.g., U.S. Pat. No. 7,190,851). Typically, the waveguide surface is functionalized by a lipid bilayer. Non-specific binding of the reporter (fluorescent) antibody is determined by the addition of control serum and then the antibody to the functional surface. Very little non-specific binding has been observed with the assays evaluated so far. Subsequently, the antigen (in serum) is added followed by a reporter antibody. Other labeled recognition molecules could be used where available. A specific signal due to antigen binding is determined. This assay approach has been validated for mycobacterial Lipoarabinomannan and has achieved less than about 10 femtomolar sensitivities of detection. This exceptional sensitivity is potentially because of the optimal orientation of the antigen in the bilayer. Several epitopes are recognized by the polyclonal reporter antibody that was raised in an animal and potentially saw the antigen in a similar conformation. Detection of lipopolysacharide from E. coli has also been accomplished. It is expected that sensitivities in the range of about 1 pM may be achieved for detection of the antigen. This platform is applicable to all lipophilic PAMPs including but not limited to flagellin, lipid A, phenolic glycolipid, cardiolipin, di-acyl lipopeptide, tri-acyl lipopetide, and peptidoglycan.

2) Insertion in the lipid bilayer allows for antigen presentation in the same orientation/conformation as can be expected in vivo. This can be exploited for antigen presentation for generation of antibodies in combinatorial approaches such as phage display.

3) Lipid insertion is a novel way of antigen concentration. Flowing complex samples like serum containing lipophilic PAMPs can allow for the selective concentration of PAMPs on the lipid surface. The antigen can then be extracted for other functional use (immunoassays etc) or can be used for techniques such as mass spectroscopy.

A novel mechanism of antigen presentation to the host cell innate immune receptors has been discovered. This changes the central dogma of toll-like receptor functionality. The novel applications of the technology, in addition to the basic biology clarification, are a) its use as a diagnostic platform (a sensor) for the sensitive detection of PAMPs in a single reporter fluorescence assay format; b) its use as a platform for antigen presentation for efficient combinatorial screening and c) its use as a platform for lipophilic antigen concentration. It should be noted that the sensitive detection of this broad class of lipid biomarkers is traditionally very difficult primarily because of the difficulty to raise two or more antibodies to these molecules that recognize orthogonal epitopes, which is required for the conventional sandwich immunoassay. This approach requires only one antibody (recognition molecule).

The present invention allows for:
1. An ultrasensitive platform of pathogen detection;
2. A unique approach for antigen presentation; and,
3. A unique approach for antigen concentration.

The present invention follows the recognition the discovery of basic principles of antigen presentation in the host during invasion by a pathogen. It changes the understanding of antigen recognition by the host immune system, especially the toll-like receptors.

The invention is an application of this discovery to the development of an ultrasensitive single reporter sensing platform for pathogens.

The invention is an application of this discovery to achieve efficient selection of recognition ligands by combinatorial methods such as phage display.

The invention is an application of this discovery to achieve efficient antigen concentration for use in techniques such as mass spectroscopy.

### BRIEF DESCRIPTION OF THE DRAWINGS

FIG. 1.1 shows a schematic representation of a sandwich immunoassay on a waveguide surface and FIG. 1.2 shows a single reporter assay that exploits the chemistry of the biomarker for selective capture on a supported lipid biolayer.

FIG. 2 shows structures of key biomolecules.

FIG. 3 shows a graph illustrating typical detection of LAM in patient urine with an internal standard for extrapolation of the concentration of the biomarker in the unknown sample and a plot of the summary of the detection of LAM in sixteen patient urine studies in a blind study.

FIG. 4 shows a graph illustrating detection of: (1) 10 femtomolar LAM; and, (2) 1 picomolar ESAT6 in serum using a single-reporter strategy on a waveguide-based sensor.

FIG. 5 shows: (1) a schematic representation of an assay; and, (2) typical spectra obtained for simultaneous detection of 10 picomolar streptavidin and 100 picomolar each of protective antigen and lethal factor within 15 minutes.

FIG. 6 shows a graph illustrating detection of 500 picomolar lipomannan of Mycobacterium bovis using a single reporter.

### DETAILED DESCRIPTION OF THE INVENTION

An aspect of the present invention is based on the belief that the lipid component in, e.g., PAMPs, has a significant role in the recognition by the host innate immune response. Here, data is presented to support the idea that the lipid component of PAMPs inserts itself into the host cell membrane and this is the molecular conformation in which the pathogen is presented to the TLRs. This observation has now been used to develop a sensor for PAMPs based on lipid insertion. In the present invention, a synthetic lipid bilayer is generally used to mimic the surface properties of natural biological components.

The sensitive insertion of two Pathogen Associated Recognition Motifs (PAMPs) in lipid bilayers as a novel mechanism by which antigens are presented to toll-like receptors in the body has now been documented. This same lipid insertion of PAMPs and related lipid biomarkers also can serve as a detection platform where the bilayer serves to “capture” the marker and subsequent exposure to a dye-labeled recognition molecule (e.g., antibody) can report this binding. Ultra-sensitive and specific assays for bacterial lipopolysacharide and mycobacterial lipoarabinomannan have been developed using this platform. These are single-reporter fluorescence based assays. Other PAMPs such as flagellin, peptidoglycan (in progress), lipid A, di- and tri-acyl lipids and cardiolipin may be expected to be captured and assayed using this approach. This pathway can be applied to all lipophilic targets for achieving sensitive detection. It may also be used for efficient antigen presentation for selection using combinatorial approaches such as phage display.

It has been found that lipophilic Pathogen Associated Molecular Patterns (PAMPs) insert themselves into a lipid bilayer. This is potentially how they are presented to the host cell and to innate immune mediators such as toll-like receptors. This changes the central dogma of innate immune recognition and perception of antigen presentation. The resultant capture approach of moieties containing a lipophilic domain is expected to be applicable to all PAMPs as well as many other molecules that are structurally and chemically similar (i.e., those that possess amphiphiles having hydrophobic groups).

The lipid components that can be used for forming the synthetic lipid bilayers in the present invention are generally described in the literature. Generally, these components are phospholipids, such as, for example, phosphatidylcholines, phosphatidylethanolamines, phosphatidylglycerols, phosphatidylserines, phosphatidic acids, phosphatidylinositols or sphingolipids. They may have lipid portions of varying length or may be of the same length. Formation of a synthetic lipid bilayer upon a surface, e.g., a waveguide surface, can be accomplished by vesicle fusion, a process well known to those skilled in the art. Generally, the lipid bilayer is formed on a solid support surface such as a waveguide or the like.

As for what may be captured or isolated from a sample, the number of lipophilic biomarkers that can be detected using this approach continues to expand.

Lipophilic pathogen activated molecular patterns (PAMPs) insert into the lipid bilayer on a cell membrane to facilitate antigen recognition by the innate immune response receptors. This changes the basic understanding of antigen recognition by the innate immune system. We have been able to adapt this observation to develop a sensor platform for the ultra-sensitive and specific detection of PAMPs associated with disease. To date, this technology has been validated with Lipoarabinomannan (LAM) from Mycobacterium tuberculosis and lipopolysacharide (LPS), associated with gram-negative bacteria. It may be that this platform is extendable to all lipophilic targets associated with pathogens and thus, is the basis of a very simple and specific sensing platform. In addition, new applications for this technology in the selection of recognition ligands by mass spectroscopy have been identified.

Using a device such as described in U.S. Pat. No. 7,190,851, this platform has been adapted to the sensitive detection of biomarkers associated with influenza (Kale, et. al., 2008), breast cancer (Mukundan, H., et. al., Bioconjugate Chemistry, in revision; Mukundan, H., et al, Sensors and Actuators, in revision) and tuberculosis (Mukundan, H. et. al., Abstract, Keystone Symposia on Tuberculosis, Vancouver, 2007; Keystone Symposia on Emerging Infections and Drug-Resistant Organisms, Bangkok, 2008). Quantitative detection of breast cancer and tuberculosis biomarkers has been achieved in complex biological fluids such as serum, nipple aspirate fluid and urine. No single biomarker can accurately detect disease. To transition this assay to a point-of-care detection format, we have also developed a multi-channel waveguide chip that will allow for the rapid (15 min.) simultaneous quantitative detection of a suite of biomarkers in a single biological sample with exquisite sensitivity.

More recently, work on mimicking TLR recognition of PAMPs on a functionalized waveguide surface to develop a sickness sensor that can allow for the pre-symptomatic diagnosis of infection has been conducted. To this effect, a simple (requires only one recognition molecule) and sensitive assay for a surface mycolic acid of Mycobacterium tuberculosis, lipoarabinomannan, on a functionalized waveguide surface, has been developed. The assay is ultra-sensitive (LOD<10 fM), rapid (15 minutes to result) and inexpensive. This approach was based on the lipid insertion of PAMPs into the host cell membrane. A lipid bilayer was coated directly onto the surface of a waveguide. This set-up was then loaded on a flow cell. Non-specific interactions of the detector antibody were determined by adding control serum (where applicable) followed by the fluorescently labeled detector antibody. Any signal because of non-specific interactions on the waveguide surface was measured. Repeat measurements were made to ensure saturation of non-specific interactions on the lipid bilayer surface. The sample containing the antigen was subsequently added. The efficacy of the assay was evaluated in buffers and in complex fluids such as serum. Following a five-minute incubation, unbound antigen was washed away and the detector antibody was again injected into the flow cell. Any measured signal was due to the specific interaction of the detector with the antigen. The antigen was bound to the cell membrane by ‘lipid insertion’. While not wishing to be bound by the present explanation, it is believed that this is a universal phenomenon observed in PAMPs and hence, this approach will work equally well for the detection of all known PAMPs currently recognized by extracellular TLRs (bacterial PAMPs) as well as many virulence markers for diseases (e.g., phenolic glycolipid for M. tuberculosis). Indeed, this novel approach has been demonstrated to detect lipopolysaccaride (LPS).

It may be expected that this approach will work for the sensitive detection of multiple known bacterial PAMPs including three different TLR targets, namely lipopolysaccharide (LPS), peptidoglycan (PGN) and lipomannan (LM) in complex fluids (e.g.; serum). Lipopolysaccharide is chosen because it is a key component of virulence in several gram-negative bacteria, including Brucella Burkholderia and Francisella. Specific antibodies that recognize the LPS of Brucella are commercially available, thereby allowing a simple demonstration of the feasibility of this approach to the detection of Brucellosis in the host. Peptidoglycan and lipomannan are chosen because antibodies are currently available and they are representative of other PAMPs that are markers for important bacterial infections. This approach will be extended to other PAMPs such as diacyl- and triacyl-peptides once recognition ligands become available. In addition to demonstration of detection, the limits of detection will subsequently be determined, as will the time required for detection, and the specificity (e.g., cross-reactivity to other biomolecules likely to be present in complex fluids). An ultimate goal is a simple hand-held sensor system that uses inexpensive, single-use cassettes that can rapidly provide actionable data in the field.

### Potential Applications of the Technology:

1) Recognition ligands generated by combinatorial approaches such as phage display typically suffer from poor sensitivities when evaluated in vitro assays. This could be because the confirmation in which the antigen is presented for ligand selection is not the same as is evidenced in vivo. It is believed that lipid insertion presents the antigen in the same conformation as it is seen by the host immune system in vivo. Therefore, lipid insertion presents a potential novel strategy that can be exploited in combinatorial approaches for the selection of antibodies and other recognition ligands that are more likely to have better efficacy in actual assays.
2) Concentration of antigens is a critical challenge for many bio-applications such as mass spectroscopy. Lipid insertion offers a novel way of concentration of lipophilic targets such as PAMPs before they are presented for mass spectroscopy. It is believed that this will allow for extensive clarification of complex samples and highly improve resolution of such techniques. Such concentration can also be extended to other applications such as microscopy and the like.
3) Mycobacterium bovis is a near neighbor of Mycobacterium tuberculosis that affects cattle. Bovine tuberculosis is a disease which causes significant economic losses to the United States and especially, to New Mexico. The surface mycolic acid of M. bovis, Lipomannan, is significantly similar in structure to LAM and hence, it is envisioned that this detection approach will work for Lipomannan as well. Development of a rapid diagnostic for the disease using this approach is another goal and transition of it to a handheld format that has previously developed for cholera. This may provide an effective rapid diagnostic for the disease.
4) PAMPs are extremely conserved in evolution. Hence, the present method has potential applications to the detection of pathogens associated with several discrepant plant and animal diseases such as paratuberculosis, Brucellosis and the like.

The following description expands on the specific target of Mycobacterium bovis.

Bovine tuberculosis (TB) is associated with significant economic loss in the United States and elsewhere. The problem is amplified by transportation of cattle across international borders and in situations where the pathogen is endemic to wildlife in that area. The current methods for detection of the disease are expensive, unreliable, insensitive, time consuming and intensive, requiring skilled technicians and complex laboratory facilities. There is thus a critical need for a rapid, sensitive and reliable test for the disease. The use of waveguide-based optical biosensor technology can deliver precisely that. A sensitive, specific and rapid ‘cow-side’ diagnostic test of bovine TB using two pathogen-associated molecules that are secreted in the infected animal is achievable. This test has the added advantage of minimizing false-positives seen with the current skin test and eliminating non-specific results associated with other non-pathogenic mycobacterial co-infection.

Zoonotic TB, caused by Mycobascterium bovis, is a serious concern to the livestock industry. In 1990, fifty million cattle were infected worldwide, and the annual economic loss was estimated at ˜3 billion dollars. Since 1990, the incidence of the disease has increased by ˜18% per year. Movement of cattle between livestock farms, markets, abattoirs and across borders is considered the major cause for spread of zoonotic TB. A positive identification of the disease in a single member of a herd results in the culling/slaughter of that entire herd of cattle. Recently, the culling of an infected herd in New Mexico cost $30 million. Vaccination against M. bovis is only partially effective, and is expensive. An additional complication is that M. bovis can infect humans.

Current diagnostic strategies for bovine TB are time consuming, expensive and inaccurate. M. bovis is a member of the mycobacterial family and shares a unique lipid-rich cell wall, which makes it very difficult to culture, stain or characterize. In fact, the pathogen replicates only once in the every 12-20 hours in culture, and hence, formation of visual colonies can take several weeks. One of the major issues with the disease is that the pathogen can persist in the infected cow for several months, causing a chronic wasting illness that is hard to diagnose. Indeed, the most commonly used tuberculin skin test takes 2-3 days to result, and is associated with a 50-75% sensitivity and 90-95% specificity. It is widely appreciated that measuring redness and induration on a cow, especially a brown cow, is qualitative and not conclusive. Therefore, results are confirmed post-mortem by bacterial culture, a process requiring 4-6 weeks under laboratory conditions with experienced workers. M. bovis does not grow well in the typical selective media used for Mycobacteria, which gives rise to false negatives, further complicating inference of results. Newer technologies like interferon based assays and antibody tests are comparable in sensitivity to the inter-dermal tuberculin test but are expensive and their complexity requires specialized and trained personnel. A false positive can result in the unnecessary culling of an entire herd, resulting in extensive economic costs.

Bovine TB is extremely prevalent in South America, the source of steers for most of South Western United States (US). The state of New Mexico recently lost its TB-free accreditation, and will soon be required to test all animals from the affected counties before allowing further dispersion within the US and elsewhere. It is estimated that this additional testing will cost producers in New Mexico $6 million per year. It is possible that California could soon also lose its TB-free status thereby requiring additional testing with an even higher cost to producers. In Michigan and Minnesota, the disease is endemic to wildlife, resulting in a more chronic and persistent problem. All animals imported from New Mexico (˜350,000 per year at the Santa Teresa crossing in New Mexico alone) are tested before entry into the US. However, the 85% accuracy of the tuberculin test results in additional infected animals in US herds. Thus, there is an immediate need for an inexpensive, rapid, specific, sensitive and reliable test for bovine TB, especially in states that have lost their TB-free accreditation.

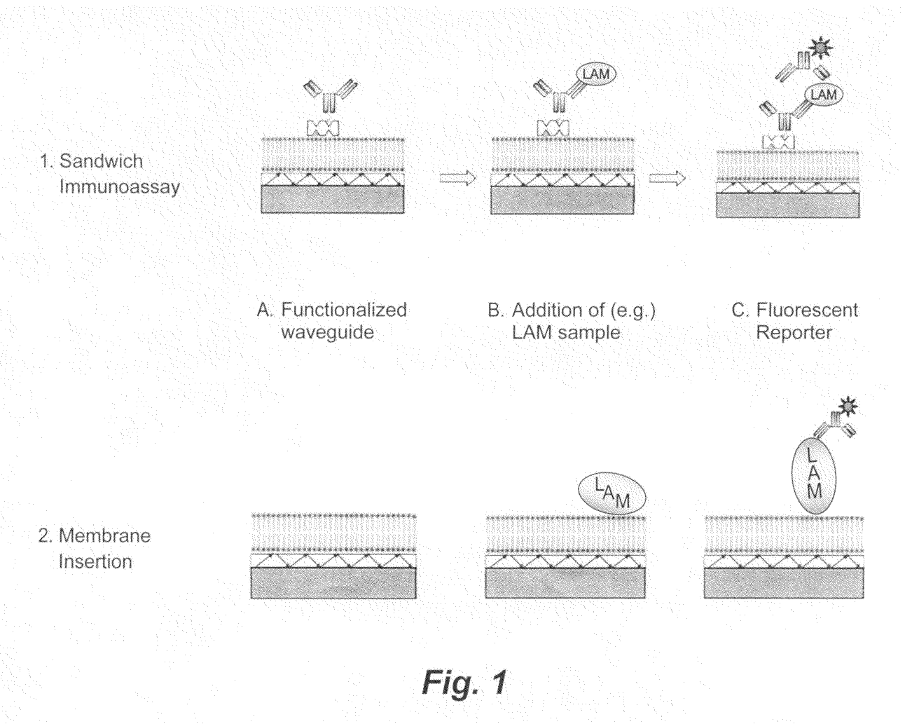

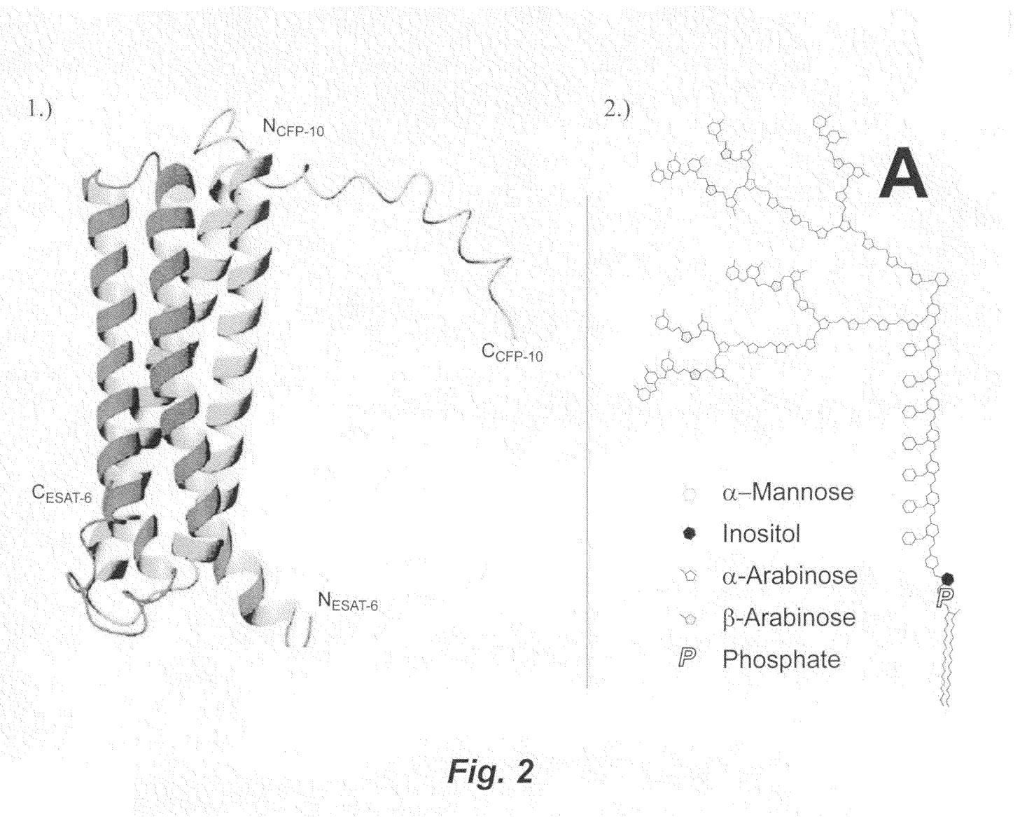

The detection strategy of the present invention exploits the superior optical properties of waveguides and the unique sensitivity of fluorescence-based immunoassays to achieve a rapid, specific and sensitive detection of disease markers. Two different transduction schemes for detection of disease biomarkers have been developed, sandwich immunoassays and single-reporter assays. A first format (FIG. 1-1) involves trapping the biomarker of interest between two specific recognition ligands, one of which is fluorescently labeled. While this approach is not an embodiment using a lipid bilayer capture approach, it has been successfully adapted to the detection of biomarkers associated with several diseases such as breast cancer, influenza, anthrax, and TB in complex biological samples such as serum and urine. The sandwich immunoassay approach, however, cannot be applied to smaller biomarkers for which two recognition ligands that can bind complimentary sites on the same target are largely unavailable. The lipid bilayer capture approach is a second strategy (FIG. 1-2) that requires only a single fluorescently labeled recognition ligand and exploits the structural motif of the biomarker for successful capture. Preliminary results from our laboratory demonstrate that this approach can allow for the detection of certain biomarkers with unprecedented sensitivity in complex biological samples.

Sensitive and rapid assays have been developed for the detection of biomarkers associated with TB in human urine. Present assays can use both transduction approaches mentioned and offer exquisite sensitivity and specificity. Mycobacterium bovis, the causative agent of bovine TB, belongs to the same bacterial family as M. tuberculosis and hence, shares significant sequence homology. Hence, it is believed that many of the transduction approaches developed for the former are easily translated to the latter. Preliminary studies show that this is indeed the case. Based on these results, it is expected that this waveguide-based biosensor technology can be adapted to the detection of M. bovis biomarkers as infected cattle with relative ease and hence develop a rapid detection strategy that will be invaluable to the agricultural and veterinary industry, while protecting our national security. Development of a two-biomarker assay for detection of M. bovis and validating feasibility in serum samples from experimentally infected animals is an important aspect of the present invention.

Several biomarkers expressed by M. tuberculosis are shared by M. bovis. Whereas detection of these antigens will allow for the specific diagnosis of the disease, the approach has been limited by the lack of sensitive and specific technology to accomplish this. The present waveguide-based biosensor is capable of the ultra-sensitive and rapid detection of pathogen-specific biomarkers in complex fluids with minimal non-specific interactions. Indeed, this platform has been successfully adapted to the detection of TB-specific biomarkers in actual patient urine.

For sandwich immunoassays (FIG. 1-1), the waveguide is functionalized with a saline-based self-assembled monolayer, and a capture antibody is entrapped on the surface using standard biotin-avidin chemistry. The use of SAMs increases the robustness of the assay and minimizes non-specific interactions associated with complex samples. A biotinylated antibody is then attached to this surface, for ‘capture’ of the desired antigen.

Upon addition of the sample, the antigen, if present, will bind to the attached capture antibody. Subsequent addition of a fluorescently labeled reporter antibody results in a specific binding signal, which is measured via a spectrometer interface. In all experiments, the waveguide-associated background is recorded, which is an intrinsic measure of impurities associated with the waveguide itself. In addition, non-specific binding associated with the fluorescently labeled reporter antibody and control samples (e.g. normal serum or urine) is measured, thereby confirming the validity of the observed measurement. In experiments where a patient sample is being evaluated, a measurement of a purified ‘standard’ lot of the biomarker in question is also performed. The signal intensity from this measurement confirms assay function and also allows for the extrapolation of the concentration of the biomarker in the unknown sample. The assay, following waveguide functionalization and set up, takes 15 minutes to completion.

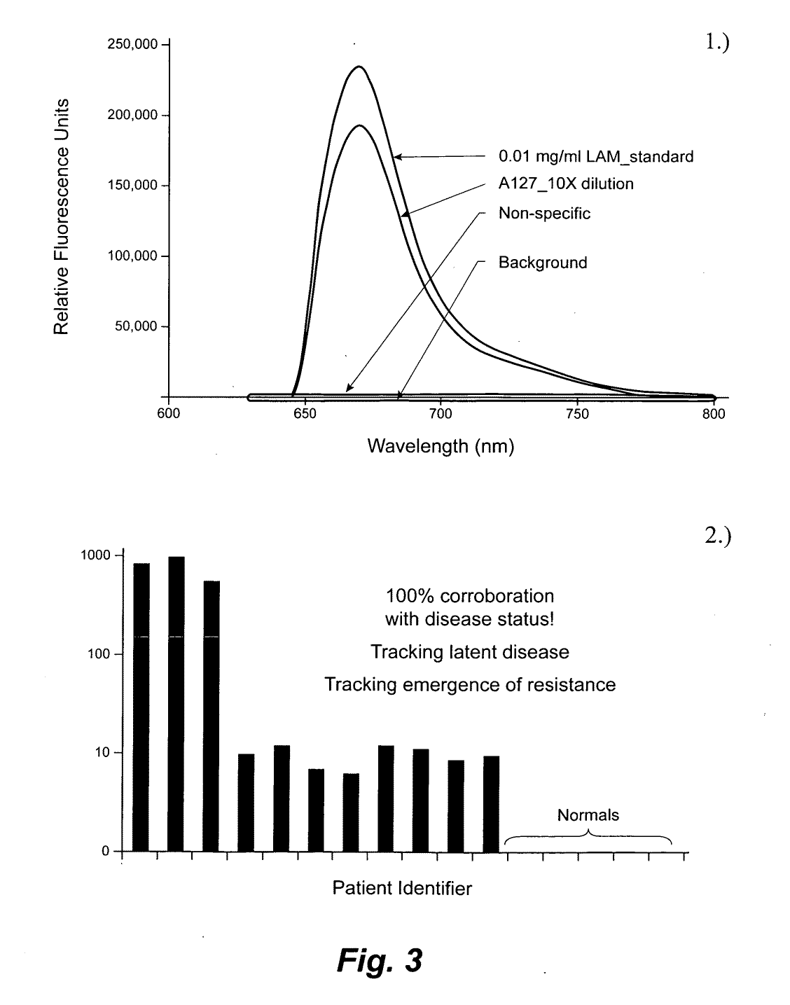

One of the major virulence factors in human TB for which a process has been adapted via a sandwich assay is lipoarabinamannan (LAM) (FIG. 2-2), a cell wall mycolic acid that contributes to host invasion by allowing the pathogen to evade phagocytosis. FIG. 3-1 demonstrates the detection of LAM in infected patient urine (patient identifier A127, blind study) and the measurement of the internal ‘standard’ in the same experiment. FIG. 3-2 demonstrates the summary results of similar measurements of LAM in sixteen different patient samples from a NIH-operated research hospital in Masan, South Korea. This was a blind-study, with no prior knowledge of the disease status of the patient. As shown on FIG. 3-2, 100% corroboration with disease status was obtained using this sandwich immunoassay platform. The limit of detection of the sandwich immunoassay platform for the LAM assay is 1 pM in serum. Ultra-sensitive assays have been developed for other biomarkers (e.g. ESAT6 (FIG. 2-1), antigen 85 complex) using the sandwich immunoassay platform and obtained excellent sensitivities in either case.

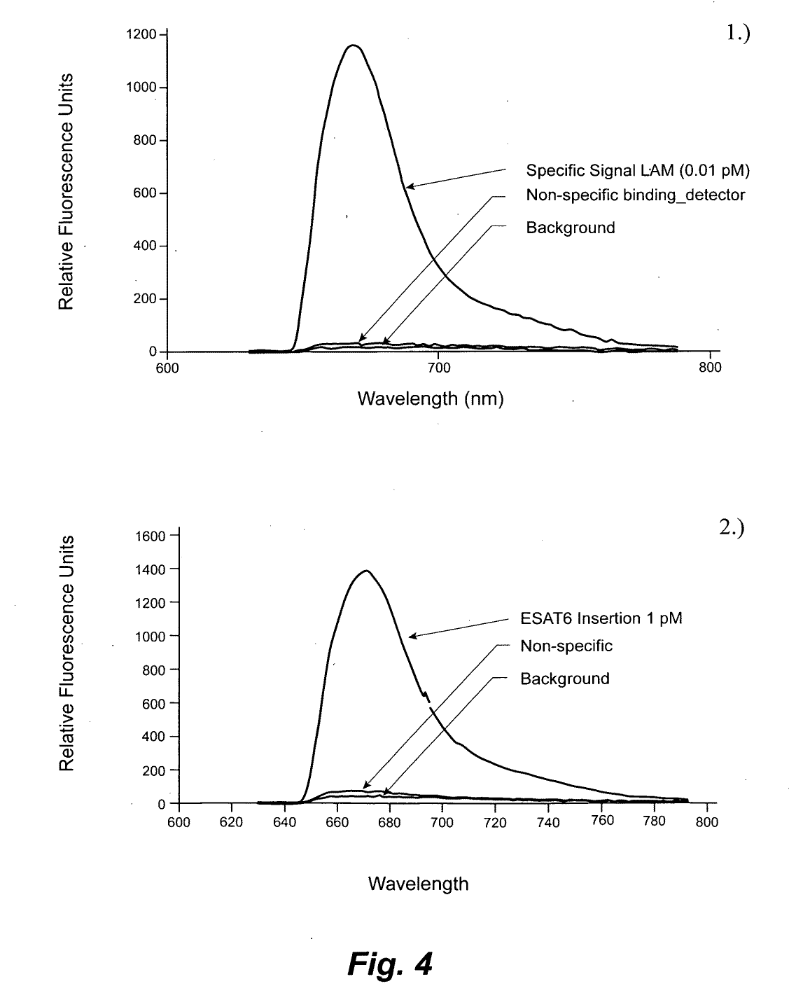

In addition to the sandwich immunoassay platform, a single reporter strategy for biomarker detection has been developed using the waveguide platform (FIG. 1-2). We have successfully adapted the LAM assay to this format. This approach relies on the spontaneous concentration (insertion) of molecules with specific chemistries into a supported lipid bilayer, thereby mimicking their in vivo association with cell membranes. Presentation of these molecules to antibodies that recognize them in a near in vivo conformation allows for binding with unprecedented sensitivities. Using this approach, femtomolar concentrations of LAM in serum can be detected (FIG. 4-1), which is unprecedented in any immunoassay platform for such an antigen. Current work is focused on optimizing the assay with urine samples and evaluating it on infected human samples. This approach allows for the rapid (15 minutes), ultra-sensitive (<10-100 femtomolar) and specific detection of LAM in complex samples using a single fluorescently labeled-reporter. This assay has been adapted to the detection of ESAT6 (FIG. 4-2) as well, a virulence factor with porin function during host invasion. Exploiting this membrane association, ESAT6 can be detected with a sensitivity of <1 pM at 37° C. in 30 minutes. ESAT6 is a very small virulence factor and although it is critical to the pathology of M. tuberculosis, no direct assays for the biomarker are currently available. The hose immune response to ESAT6 is, in fact, the basis of several alternative tests for TB such as the QuantiFERON γ test and the EliSpot® test. These tests require extensive sample preparation and handling and are affected by differential host immune responses.

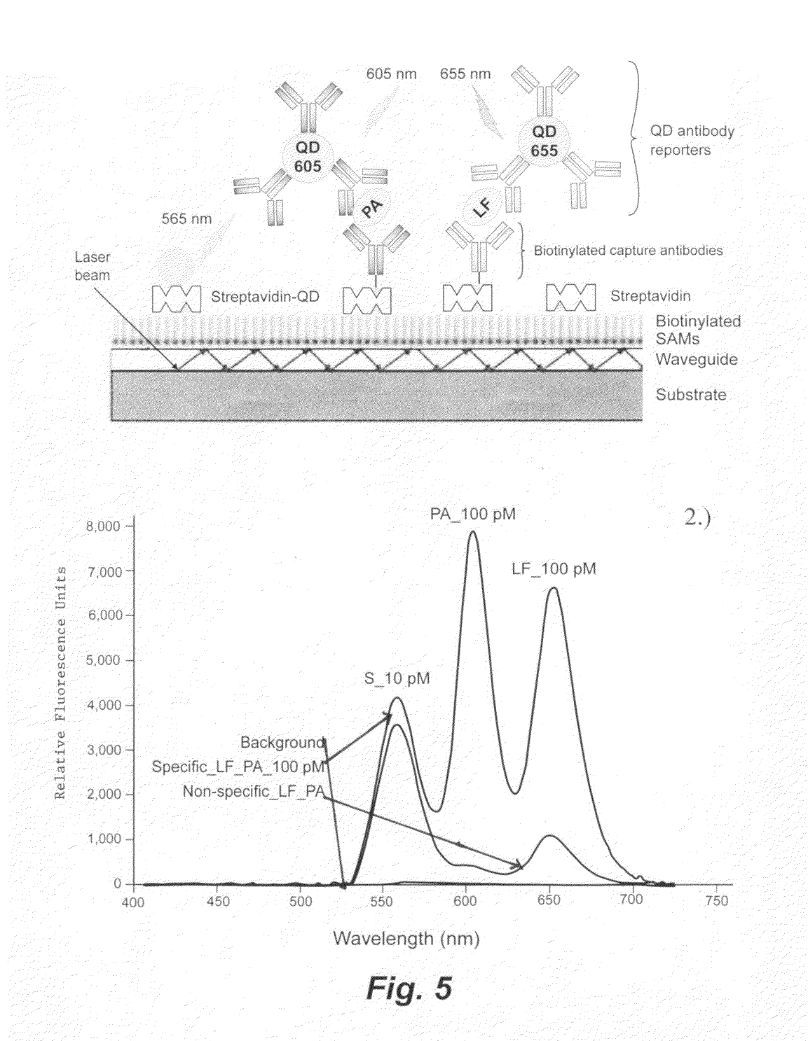

No single biomarker can accurately predict disease. Hence, work has been on development of a multiplex assay platform for the simultaneous detection of a limited suite of such molecules. This strategy (FIG. 5-1) uses photostable and tunable quantum dots as the fluorescence reporter. A conjugation process has been successfully developed for quantum dots and have also validated our multiplex platform for the simultaneous detection of two lethal toxins of Bacillus anthracis in serum (FIG. 5-2). In addition, the present invention has lead to the design, fabrication and evaluation of multichannel waveguides (FIG. 5-3) for the simultaneous detection of three samples in quadruplicate within 15-30 minutes, thereby increasing the throughput of such an assay format. Translation of this technology to the simultaneous detection of TB biomarkers may be accomplished with relative ease.

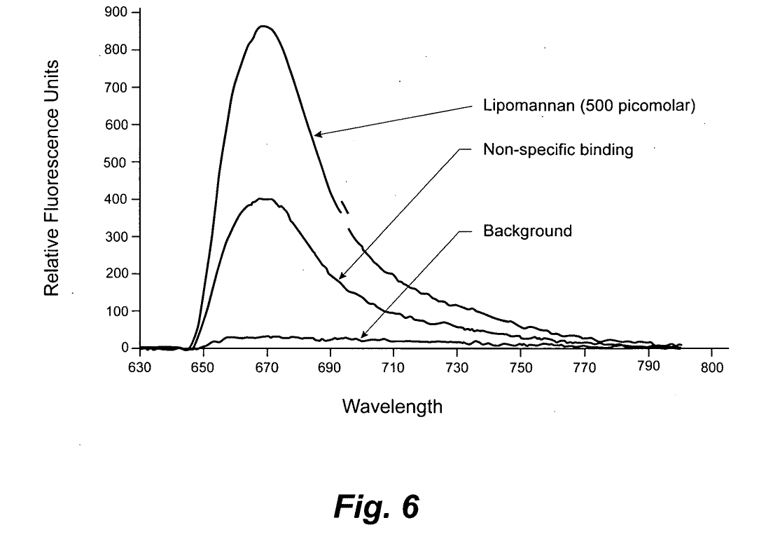

M. tuberculosis and M. bovis belong to the same genera and hence, share a significant sequence homology. Indeed, many of the biomarkers are common to all Mycobacteria, albeit with small structural modifications that may influence pathogenecity. M. tuberculosis LAM bears a strong structural similarity to its counterpart on the M. bovis cell wall, namely lipomannan (LM) (FIG. 2-3). Therefore, it follows to reason that a polyclonal antibody for LAM is likely to bind LM, albeit with a lower sensitivity. Preliminary studies (FIG. 6) show that this is true. M. bovis LM has been detected using both the sandwich immunoassay and single-reporter approach, with a sensitivity of ˜1 pM, within 15 minutes in bovine serum. These results clearly validate the feasibility of this approach for the detection of ultra low concentrations LM in serum. Final optimization of this assay will require the development of M. bovis LM-specific antibody.

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### REFERENCES

1. Uematsu S., Akira S, 2008, Toll-Like Receptors (TLRs) and their Ligands. Handb Exp Pharmacol (183): 1-20
2. Kale R et al., 2008, Detection of Intact Influenza Viruses using Biotinylated Biantennary S-Sialosides; JACS (130):8169-8171.
3. Mukundan H et al, 2008, Planar Optical Waveguide-based Biosensor for the Quantitative Detection of Tumor Markers, Sensors and actuators, in revision.
4. Mukundan H et al, 2008, Optimizing a Waveguide-based Sandwich Immunoassay for Tumor Biomarkers: Evaluating fluorescent labels and functional surfaces. Bioconjugate Chemistry, In revision.
5. Mukundan H et al, 2007, Waveguide-based Optical Biosensor for Detection of Tuberculosis-Specific Antigens in Patient Urine. Abstract for poster presentation, Keystone Symposia on Tuberculosis, Vancouver.

What is claimed is:

1. A method of detecting the presence of one or more target moieties within a sample comprising:
exposing a lipid bilayer to a sample including one or more target moieties characterized by having a lipophilic portion of sufficient size and chemical composition whereby one or more of said target moieties inserts into said lipid bilayer; and,
breaking the lipid bilayer and passing the contents to a mass spectrometer for analysis of the one or more of said target moieties inserted into said lipid bilayer.

2. The method of claim 1 wherein said one or more target moieties is selected from pathogen associated molecular patterns.

3. The method of claim 1 wherein said lipid bilayer is upon a functionalized waveguide surface.

4. The method of claim 1 wherein said one or more target moieties within a sample includes Mycobacterium bovis.

5. The method of claim 2 wherein said pathogen associated molecular patterns include lipoarabinomannan (LMO) and lipopolysaccharide (LPS).

6. The method of claim 1 wherein said lipid bilayer is a synthetic lipid bilayer.

7. A method of detecting the presence of one or more target moieties within a sample comprising:
exposing a lipid bilayer to a sample including one or more target moieties characterized by having a lipophilic portion of sufficient size and chemical composition
exposing said lipid bilayer including said one or more inserted target moieties to one or more labeled moieties, such labeled moieties having both a defined binding affinity for at least one of said one or more target moieties and a detectable label in the event of binding between a labeled moiety and an inserted target moiety; and,
examining said lipid bilayer, following said exposure to one or more labeled moieties, for the presence of a target moiety bound to a labeled moiety.

8. The method of claim 7 wherein said one or more target moieties is selected from pathogen associated molecular patterns.

9. The method of claim 7 wherein said lipid bilayer is upon a functionalized waveguide surface.

10. The method of claim 8 wherein said pathogen associated molecular patterns include lipoarabinomannan (LMO) and lipopolysaccharide (LPS).

11. The method of claim 7 wherein said one or more target moieties within a sample includes Mycobacterium bovis.

12. The method of claim 7 wherein said lipid bilayer is a synthetic lipid bilayer.
